(19) United States
(12) Patent Application Publication
(10) Pub. No.: US 2024/0130975 A1
(43) Pub. Date: Apr. 25, 2024

(54) DRUG DELIVERY IN NEGATIVE POISSON`S RATIO STRUCTURES

(71) Applicant: Joon Bu Park, Las Vegas, NV (US)

(72) Inventor: Joon Bu Park, Las Vegas, NV (US)

(21) Appl. No.: 17/971,015

(22) Filed: Oct. 20, 2022

(51) Int. Cl. A61K 9/48; A61J 3/07

(52) U.S. Cl. CPC A61K 9/4808; A61J 3/07; A61K 9/4875

(57) ABSTRACT

A drug delivery device includes a foam body having a negative Poisson's ratio, the foam body formed of a material configured to dissolve in a biological fluid; and a liquid drug contained in cells of the foam body, the drug exerting a pressure on walls of the cells that is above atmospheric pressure.

### BACKGROUND

The present disclosure relates generally to drug delivery devices.

### SUMMARY

We describe here drug delivery devices that include materials having a negative Poisson's ratio (“NPR materials”) that can provide various advantages over the use of positive Poisson's ratio-only materials (“PPR materials) for drug delivery. For example, in some implementations, the negative Poisson's ratio materials can provide improved mechanical stability to the drug delivery structures, allowing for higher internal pressures. In some implementations, the drug delivery structures can be lighter weight because of the inclusion of NPR materials. In some implementations, the NPR materials can provide improved stress response characteristics to the drug delivery structures. In some implementations, the drug delivery devices can be handled and transferred at body temperature and/or pressure, allowing the stored drug to be delivered within the body for use in reactions. In some implementations, the porous nature of NPR materials is conducive to drug delivery. Stored drugs can include antihistamines (e.g., chlorpheniramine maleate), decongestants (e.g., pseudoephedrine hydrochloride), insulin, heparin, vaccines, pain killers (e.g., morphine), or other drug types.

In an aspect, a drug delivery device includes a foam body having a negative Poisson's ratio, the foam body formed of a material configured to dissolve in a biological fluid; and a liquid drug contained in cells of the foam body, the drug exerting a pressure on walls of the cells that is above atmospheric pressure.

Embodiments can include one or any combination of two or more of the following features.

The drug includes an antihistamine.

The foam body includes a re-entrant cell structure.

The foam body includes a disordered, polydisperse foam.

The foam body is a microsphere or a nanosphere.

The foam body has a dimension of less than 100 microns.

The foam body has a dimension of less than 500 nanometers.

At least some of the cells of the foam body are closed cells.

The liquid drug includes a first liquid drug, and the drug delivery device includes a second liquid drug, wherein the first liquid drug is contained in a first set of cells of the foam body and the second liquid drug is contained in a second set of cells of the foam body, the cells of the first set located closer to an outer surface of the foam body than the cells of the second set.

The walls of the cells include first walls having a first thickness and second walls having a second thickness different from the first thickness.

The drug delivery device includes a coating covering the foam body, the coating dissolvable in the biological fluid and having a positive Poisson's ratio.

In an aspect, combinable with any of the previous aspects, a drug delivery apparatus includes a capsule defining an interior space, the capsule including a material having a positive Poisson's ratio and configured to dissolve in a biological fluid; and a powder contained in the interior space of the capsule, the powder including drug delivery devices. Each drug delivery device includes a foam body having a negative Poisson's ratio, the foam body formed of a material configured to dissolve in a biological fluid; and a liquid drug contained in cells of the foam body, the drug exerting a pressure on walls of the cells that is above atmospheric pressure.

Embodiments can include one or any combination of two or more of the following features.

The drug delivery device includes first drug delivery devices each including a first foam body, in which the first foam bodies have cells including walls of a first thickness; and second drug delivery devices each including a second foam body, in which the second foam bodies have cells including walls of a second thickness different from the first thickness.

The drug delivery device includes first drug delivery devices each including a first foam body and a first liquid drug contained in cells of the first foam body; a second drug delivery devices each including a second foam body and a second liquid drug contained in cells of the second foam body, the second liquid drug different from the first liquid drug.

The powder includes drug delivery devices of different sizes.

In an aspect, combinable with any of the previous aspects, a method of making a drug delivery apparatus includes providing a first body including a precursor material; applying a stimulus to the precursor material, the stimulus causing the precursor material to form a closed-cell porous foam structure having a negative Poisson's ratio; and encapsulating a fluid drug in the closed-cell porous foam structure such that the fluid drug exerts a pressure on walls of cells of the foam structure that is above atmospheric pressure, in which the porous foam structure is formed of a material configured to dissolve in a biological fluid.

Embodiments can include one or any combination of two or more of the following features.

The stimulus includes at least one of heat or pressure.

The drug includes an antihistamine.

The method includes subsequent to applying the stimulus, subsequent to applying the stimulus, dividing the closed-cell porous foam structure into a plurality of drug delivery devices, each drug delivery device having a dimension less than 100 microns.

The method includes forming a coating having a positive Poisson's ratio over the closed-cell porous foam structure.

Implementations of this and other described apparatuses can have any one or more of at least the following characteristics.

Other implementations are also within the scope of the claims.

### DESCRIPTION OF DRAWINGS

FIGS. 1A and 1B are diagrams of NPR drug delivery devices.

FIG. 2 is a diagram illustrating force responses of example NPR and PPR materials.

FIG. 3 is a diagram illustrating a drug being injected into NPR drug delivery devices.

FIGS. 4A-4D are diagrams illustrating an example of a method of making drug-encapsulating NPR drug delivery devices.

FIGS. 5A-5B are diagrams illustrating an example of a method of making drug-encapsulating NPR drug delivery devices with a PPR coating.

### DETAILED DESCRIPTION

This disclosure describes drug delivery using materials having a negative Poisson's ratio (“NPR materials”), sometimes referred to as auxetic materials. In some implementations, an NPR foam body or composite NPR-positive Poisson's ratio (PPR) foam body contains a drug such as an antihistamine, decongestant, insulin, heparin, vaccine, pain killer, etc. The NPR composition of the foam body can facilitate reduced weight and improved mechanical stability (e.g., improved stress response) during drug storage and delivery.

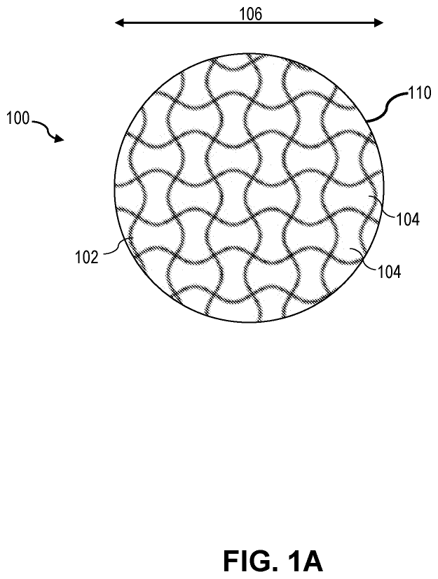

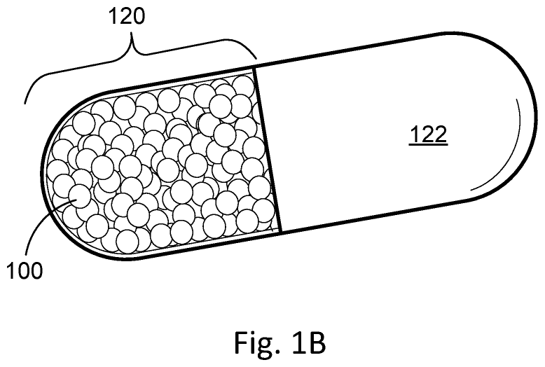

Referring to FIG. 1A, an example drug delivery device 100 has a porous (e.g., foam) structure 102 that has a negative Poisson's ratio. The porous structure 102 defines cells 104, at least some of which are “closed,” meaning that the cells 104 are fully enclosed by the porous structure 102. As discussed below, the structure of the cells 104 causes the porous foam structure 102 to exhibit a negative Poisson's ratio. A fluid (e.g., liquid) drug such as an antihistamine (e.g., chlorpheniramine maleate), decongestant, insulin, heparin, vaccine, pain killer, or other fluid drug is encapsulated in the cells 104. The porous foam structure 102 of the drug delivery device 100 is filled with a quantity of the fluid drug such that the drug exerts a pressure on the walls of the cells 102 (referred to as a positive pressure). For example, the drug can exert a pressure on the walls of the cells 102 that is higher than atmospheric pressure (1 atm) at room temperature. In the illustrated example, the porous foam structure 102 is coated with a coating 110 that has a positive Poisson's ratio (a “PPR coating”). In some examples, the porous foam structure 102 is uncoated or is coated with a coating that is an NPR material.

The cellular pattern of the porous structure 102 provides a negative Poisson's ratio (auxeticity) to the drug delivery device 100 as a whole, and the porous structure 102 can be described as a whole as an NPR material. An NPR material is a material that has a Poisson's ratio that is less than zero, such that when the material experiences a positive strain along one axis (e.g., when the material is stretched), the strain in the material along the two perpendicular axes is also positive (e.g., the material expands in cross-section). Conversely, when the material experiences a negative strain along one axis (e.g., when the material is compressed), the strain in the material along a perpendicular axis is also negative (e.g., the material compresses along the perpendicular axis). By contrast, a material with a PPR material has a Poisson's ratio that is greater than zero. When a PPR material experiences a positive strain along one axis (e.g., when the material is stretched), the strain in the material along the two perpendicular axes is negative (e.g., the material compresses in cross-section), and vice versa.

The NPR characteristic of the drug delivery device 100 enhances the stability and capabilities of the drug delivery device 100. For example, in some implementations the drug encapsulated in the porous structure 102 is pressurized at a positive pressure with respect to an ambient pressure external to the drug delivery device 100. Internally, this pressure can equalize between adjacent cells. For the drug delivery device 100 as a whole, the pressure exerts an outward force. However, because the NPR drug delivery device 100 compresses along its axes in response to compressive forces along corresponding orthogonal axes, the drug delivery device 100 is stable in the presence of this pressure. For example, in some implementations the drug delivery device 100 can be more stable than an analogously sized PPR device for drug delivery, allowing for higher internal drug pressures to be maintained. In some implementations, the room-temperature drug pressure in at least some cells of the drug delivery device 100 is at least 10 psig, at least 100 psig, at least 500 psig, at least 1000 psig, or at least 5000 psig.

In addition, NPR materials that form the drug delivery device 100 can be lightweight, e.g., compared to other materials/systems capable of storing comparable amounts of drugs. Using NPR drug delivery bodies for delivery of drugs or medicines can require less material, improving overall delivery and increasing efficiency of the drug delivery device.

The porous structure 102 and the PPR coating 110 are both formed of a material that is biocompatible and that dissolves in a biological environment, e.g., in biological fluid such as fluid of a human body. For instance, the porous structure and/or the coating can be formed of gelatin, hydroxypropyl methylcellulose or hypromellose (HPMC), starch, polyvinyl acetate (PVA), or other suitable materials. The porous structure and coating are intact prior to delivery to the patient (e.g., by injection, ingestion, inhalation, or another suitable delivery method), and dissolves once delivered, thereby releasing the cell-encapsulated drug to the patient. The porous structure 102 and the coating 110 can be the same material or different materials.

In some examples, the material of the porous structure 102 is designed to dissolve slowly, thereby enabling drug release over a period of time, e.g., for a time-release delivery. For instance, the walls of the outermost cells 104 are exposed to the biological environment before the walls of the interior cells 104 and thus dissolve before the walls of the interior cells 104. As a result, the drug contained in the outermost cells is released before the drug contained in the interior cells 104. In some examples, the structure of the drug delivery device 100 is designed to promote time-release delivery of the drug. For instance, the walls of the cells can be of non-uniform thickness, e.g., some walls are thicker than others and thus dissolve more slowly such that the drug contained in the thicker-walled cells is released later than the drug contained in the thinner-walled cells. In some examples, the coating 110 is configured to dissolve rapidly, e.g., substantially immediately upon exposure to the biological environment, while the porous structure 102 is configured to dissolve more slowly, thus providing substantially immediate exposure of the porous structure 102 to the biological environment while also facilitating a slow release of the drug from the porous structure 102.

In a specific example, the drug delivery device 100 is a polydisperse and disordered re-entrant foam structure (discussed below). The re-entrant structure of the foam provides the NPR behavior. The polydisperse and disordered nature of the foam contributes to the time-release behavior in that having cells of different sizes, and arranged in a random arrangement, helps to spread out the time of delivery of the drugs from the cells of the foam.

In some implementations, the drug delivery device 100 is “microscale” in that it has at least one dimension 106 (e.g., a length, a width, a diameter, or a thickness) that is less than 100 microns. In various implementations, the drug delivery device 100 has at least one dimension 106 that is less than 50 microns, less than ten microns, less than five microns, or less than one microns. In some implementations, the drug delivery device 100 is “nanoscale,” meaning that the dimension 106 is less than 500 nanometers, less than 100 nanometers, less than 50 nanometers, or less than 10 nanometers. The dimension 106 is limited, on its low end, by fabrication capabilities and by cell sizes of the drug delivery device 100. For example, the dimension 106 can be greater than 1 nanometer, greater than 5 nanometers, greater than 10 nanometers, greater than 50 nanometers, or greater than 100 nanometers, depending on the implementation. Different sizes of drug delivery bodies can have different dissolution rates, and different dissolution rates can be desirable depending on the drug being delivered and the desired target.

Referring to FIG. 1B, in some implementations, multiple microscale or nanoscale drug delivery bodies 100 are collectively delivered as a powder-like collection 120 contained in a capsule 122, such as a standard dissolvable drug capsule. The drug delivery bodies in the powder can be uniform in size or can be of various sizes, and can have uniform cell wall thickness or varied cell wall thicknesses, e.g., to achieve a target time-release delivery profile. In an example, some drug delivery bodies in the powder can have thin-walled cells for rapid dissolution and rapid drug delivery, while other drug delivery bodies in the same powder can have thicker-walled cells for slower dissolution and later drug delivery. In an example, the drug delivery bodies in the powder can be differently sized (e.g., have different diameters), with the smaller drug delivery bodies releasing their drugs faster than the larger drug delivery bodies. The drugs contained in the various types of drug delivery bodies can be the same or different.

In some implementations, microscale NPR drug delivery bodies can exhibit resilient mechanical properties due to volume/surface area scaling. For example, in some implementations, microscale NPR drug delivery bodies can store drugs at higher pressures than can larger drug delivery bodies or than comparably-sized PPR drug delivery bodies. Moreover, in some implementations microscale drug delivery bodies dissolve more easily than macroscopic drug delivery bodies for matching total weights of the bodies (e.g., dissolve at lower temperatures and/or more quickly), improving stored drug release characteristics in applications such as targeted therapy.

In some implementations, the drug delivery device 100 is not microscale. For example, a macroscale drug delivery device can have a dimension 106 that is greater than 50 microns, such as between 50 microns and 10 centimeters, e.g., between 1 millimeter and 5 centimeters.

The shape of the drug delivery device 100 can vary depending on the implementation. In some implementations, the drug delivery device 100 is microscale and is substantially spherical, such that the drug delivery device 100 is a microsphere or a nanosphere. In some implementations, the drug delivery device 100 is elongated, such that the drug delivery device 100 is a micro-capsule/micro-tubule or nano-capsule/nano-tubule. Other shapes are also within the scope of this disclosure. For example, a shape of the drug delivery device 100 can be defined by a mold by which the drug delivery device 100 is caused to become auxetic, such that a shape of the mold is transferred to the drug delivery device 100. Based on a shape of the mold, the drug delivery device 100 can have any suitable shape, such as spherical, cuboid, cylindrical, a polyhedron (e.g., an irregular polyhedron), or another three-dimensional shape.

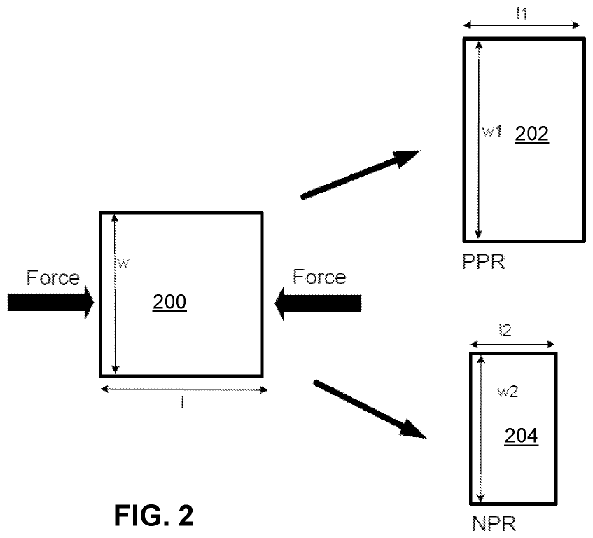

Materials with negative and positive Poisson's ratios are illustrated in FIG. 2, which depicts a hypothetical two-dimensional block of material 200 with length l and width w. If the hypothetical block of material 200 is a PPR material, when the block of material 200 is compressed along its width w, the material deforms into the shape shown as block 202. The width w₁ of block 202 is less than the width w of block 200, and the length l₁ of block 202 is greater than the length l of block 200: the material compresses along its width and expands along its length.

By contrast, if the hypothetical block of material 200 is an NPR material, when the block of material 200 is compressed along its width w, the material deforms into the shape shown as block 204. Both the width w₂ and the length l₂ of block 204 are less than the width w and length l, respectively, of block 200: the material compresses along both its width and its length.

A foam is a multi-phase composite material in which one phase is fluid (e.g., gaseous or liquid) and the one or more other phases are solid (e.g., polymeric, ceramic (e.g., a carbon-based ceramic), metallic, otherwise carbon-based, or a combination thereof). The drug delivery device 100 is a foam in which the drug is the fluid phase and the porous structure 102 is the solid phase. NPR drug delivery bodies for delivery of antihistamines and/or other drugs can be polymeric foams, ceramic foams, metallic foams, carbon foams, or combinations thereof. In general, foams can be closed-cell foams, in which each fluid cell is sealed by solid material; open-cell foams, in which each cell communicates with the outside atmosphere; or mixed, in which some cells are closed and some cells are open. For purposes of drug delivery, at least some of the cells of the porous structure 102 are closed to prevent escape of the drug contained within. In some implementations, some of the cells can be open.

An example of an NPR foam structure is a re-entrant structure, which is a foam in which the walls of the cells are concave, e.g., protruding inwards toward the interior of the cells, such as at least some of the cells 104 in FIG. 1A. In a re-entrant foam, compression applied to opposing walls of a cell will cause the other, inwardly directed walls of the cell to buckle inward further, causing the material in cross-section to compress, such that a compression occurs in all directions. Similarly, tension applied to opposing walls of a cell will cause the other, inwardly directed walls of the cell to unfold, causing the material in cross-section to expand, such that expansion occurs in all directions. NPR foams can have a Poisson's ratio of between −0.5 and 0, e.g., −0.5, −0.4, −0.3, −0.2, or −0.1. NPR foams can have an isotropic Poisson's ratio (e.g., Poisson's ratio is the same in all directions) or an anisotropic Poisson's ratio (e.g., Poisson's ratio when the foam is strained in one direction differs from Poisson's ratio when the foam is strained in a different direction).

An NPR foam can be polydisperse (e.g., the cells of the foam are not all of the same size) and/or disordered (e.g., the cells of the foam are randomly arranged, as opposed to being arranged in a regular lattice). An NPR foam can have a characteristic dimension (e.g., the size of a representative cell (e.g., average-size cell), such as the width of the cell from one wall to the opposing wall) ranging from 0.005 μm to about 3 mm, e.g., about 0.01 μm, about 0.02 μm, about 0.05 μm, about 0.1 μm, about 0.5 μm, about 1 μm, about 10 μm, about 50 μm, about 100 μm, about 500 μm, about 1 mm, about 2 mm, or about 3 mm, or any range delimited by any two of these values.

Examples of cell structures that can be realized on a macroscale or on a micro/nano scale in microscale drug delivery bodies include stackings (e.g., ABAB or AAA stackings) of a quadratic chiral lattice, inverse honeycomb, double arrow, re-entrant hexagonal, re-entrant square, and other structures that can be identified by suitable computational and/or experimental methods. Example computational methods can be found in Korner and Liebold-Ribeiro, “A systematic approach to identify cellular auxetic materials,” Smart Materials and Structures 24(2) (2015).

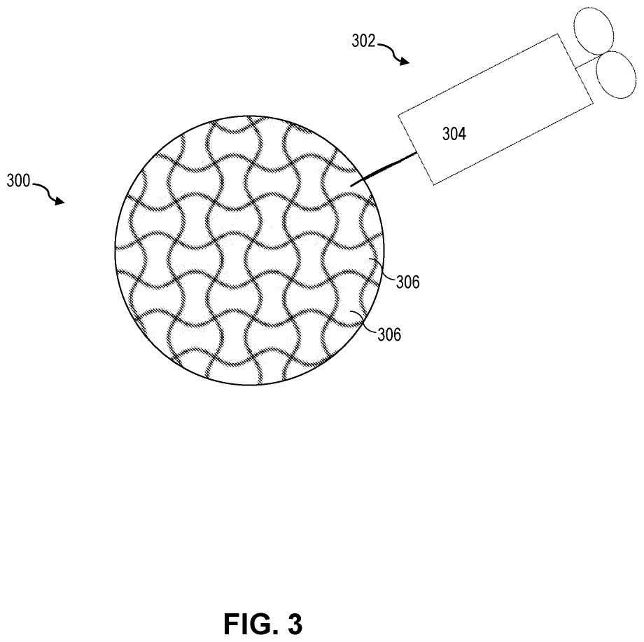

NPR drug delivery bodies (e.g., drug delivery device 100) can be injected with drugs and transferred to a target site through different drug delivery methods (e.g., oral, ophthalmic, otologic, nasal, urogenital, rectal, dermal, injection, infusion, etc.). FIG. 3 illustrates a drug delivery device 300 being injected with a drug using a syringe 304. Each cell 306 in the drug delivery device 300 can be injected with a desired amount of the drug. In some embodiments, some cells are not injected with the drug.

The drug can also be introduced into the delivery device 300 in other ways. In some examples, the drug delivery device 300 can be placed into a pool of the drug such that the drug fills the cells, e.g., by capillary action. In some examples, the drug delivery device 300 can be formed in a pool of the drug, such that as the drug delivery device 300 is formed it encapsulates the drug 304 in the cells 306.

The porous structure of drug delivery bodies, such as the porous structure 102 of the drug delivery device 100, can be formed of one or more of various materials, depending on the implementation. In some implementations, the porous structure 102 is formed at least partly of one or more biocompatible metals, ceramics, and/or polymers. For example, in some implementations the porous structure 102 is formed fully or primarily (e.g., as at least a majority of the mass of the porous structure 102) of carbon. Examples of carbon-based materials that can exhibit negative Poisson's ratio for stresses in at least some directions include porous graphite, bulk carbon nanotubes, activated carbon, collections of carbon fibers (e.g., porous carbon fibers and/or other carbon fibers), and certain carbon lattice structures (e.g., three-dimensional carbon honeycomb structures and/or fullerenes). In some implementations, a carbon-based structure is combined with one or more polymers (e.g., resin), e.g., as a carbon-fiber-reinforced polymer (CFRP) structure. Having the porous structure 102 formed substantially or entirely of bioabsorbable materials can provide benefits, because they are non-toxic and because they dissolve upon exposure to a patient's body, enabling release of the drug (e.g., time delayed release).

In some implementations, the porous structure of a drug delivery device includes a polymer and/or a metal. Examples of polymeric foams include thermoplastic polymer foams (e.g., polyester polyurethane or polyether polyurethane); viscoelastic elastomer foams; or thermosetting polymer foams such as silicone rubber. Examples of metallic foams include metallic foams based on steel (e.g., stainless steel), copper, aluminum, magnesium, zirconium, titanium (e.g., Ti₆Al₄V, TiNbZr, or unalloyed titanium), or other metals, or alloys thereof, or ceramics composed of a metal oxide (e.g., aluminum oxide, titanium oxide, or zirconium oxide).

NPR-PPR composite materials are composites that include both regions of NPR material and regions of PPR material. NPR-PPR composite materials can be laminar composites, matrix composites (e.g., metal matrix composites, polymer matrix composites, or ceramic matrix composites), particulate reinforced composites, fiber reinforced composites, or other types of composite materials. In some examples, the NPR material is the matrix phase of the composite and the PPR material is the reinforcement phase, e.g., the particulate phase or fiber phase. In some examples, the PPR material is the matrix phase of the composite and the NPR material is the reinforcement phase. NPR-PPR composite materials can form the porous structures and foam structures described in any of the implementations described herein.

NPR drug delivery bodies including closed cells to encapsulate drugs, such as the porous structure 102 of FIG. 1A, can be produced in a variety of ways. In some implementations, an initially PPR material (sometimes referred to as a “precursor material”) is converted into the NPR material. For example, a porous PPR sponge or foam can be transformed to change its structure into a structure that exhibits a negative Poisson's ratio. In some examples, NPR foams are produced by transformation of nanostructured or microstructured PPR materials, such as nanospheres, microspheres, nanotubes, microtubes, or other nano- or micro-structured materials, into a foam structure that exhibits a negative Poisson's ratio. The transformation of a PPR foam or a nanostructured or microstructured material into an NPR foam can involve thermal treatment (e.g., heating, cooling, or both), application of pressure, or a combination thereof. In some examples, PPR materials, such as PPR foams or nanostructured or microstructured PPR materials, are transformed into NPR materials by chemical processes, e.g., by using glue. In some examples, NPR materials are fabricated using micromachining or lithographic techniques, e.g., by laser micromachining or lithographic patterning of thin layers of material. In some examples, NPR materials are fabricated by additive manufacturing (e.g., three-dimensional (3D) printing) techniques, such as stereolithography, selective laser sintering, or other appropriate additive manufacturing technique.

In an example of a process, a PPR thermoplastic foam, such as an elastomeric silicone film, can be transformed into an NPR foam by compressing the PPR foam, heating the compressed foam to a temperature above its softening point, and cooling the compressed foam. In an example, a PPR foam composed of a ductile metal can be transformed into an NPR foam by uniaxially compressing the PPR foam until the foam yields, followed by uniaxial compression in other directions.

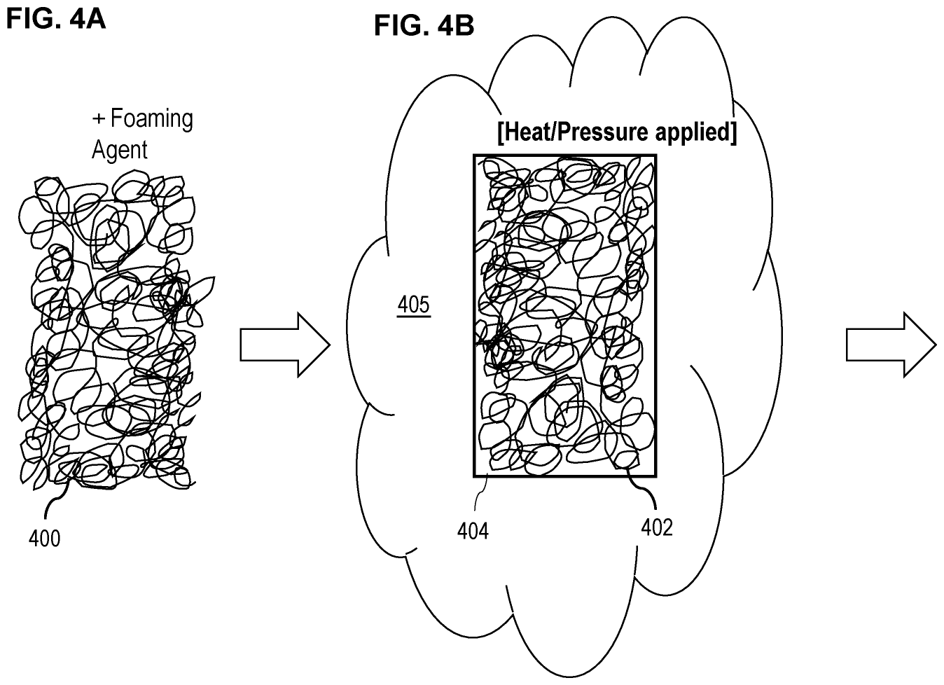

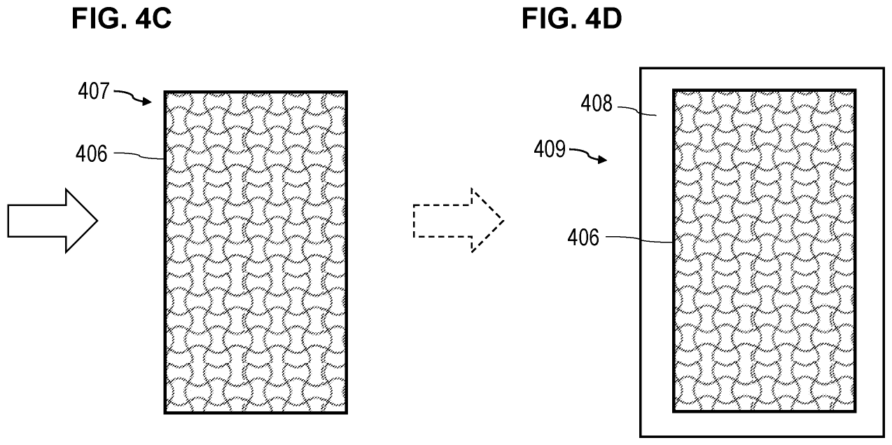

FIGS. 4A-4D illustrate an example method of making a drug delivery device, such as the drug delivery device 100. A granular or powdered material 400 (a precursor material), such as a powder of a biocompatible and biodissolvable material, is mixed with a foaming agent to form a porous material (e.g., a sponge or a foam) 402. The porous material 402 is placed into a mold 404. Pressure is applied to compress the porous material 402, and the compressed porous material 402 is heated to a temperature above its softening point. In some examples, the pressure and heating are performed in the presence of a drug. In some examples, the drug is injected into the porous material 402 after formation of the NPR material, e.g., as discussed above. The compressed, heated porous material 402 is then allowed to cool, resulting in an NPR material 406 (having a closed-cell porous structure) produced from the initially-PPR porous material 402. In some implementations, the NPR material 406 can be divided into smaller bodies (e.g., microscale bodies), e.g., forming individual drug delivery devices.

In some implementations, an NPR porous structure or composite NPR/PPR porous structure configured to store drugs is coated in a PPR material. The PPR material can act as a barrier to protect underlying NPR or composite NPR/PPR materials from corrosion, degradation, oxidation, and/or other effects of exposure. In some implementations, the PPR material can improve mechanical properties (e.g., stress response properties) of the drug delivery bodies as a whole, which can provide improved stability (e.g., improved resilience to shocks occurring during handling/transfer of the drug delivery bodies) and/or allow for higher internal drug pressures, increasing the amount of drug that can be stored in a given volume of drug delivery device. For example, because the PPR material can respond to stresses in an opposite manner than NPR materials (e.g., contracting instead of expanding), resulting forces in drug delivery bodies having the PPR material as a coating can cancel one another out, improving overall mechanical stability.

In some examples, to form a drug delivery device with a PPR coating, the NPR material 406 containing the drug is covered with an outer PPR material 408. In some examples, the PPR material 408 is adhered to the NPR material 406. In some examples, the PPR material 408 defines a cavity within which the NPR material 406 is contained. The NPR material in this arrangement can be referred to as an internal NPR portion, and the PPR coating can be referred to as an outer PPR portion. In some examples, the PPR material can be coated onto the NPR material by dip-coating, by evaporative coating, by spray-coating, or by another suitable method. In some examples, the PPR material is a preformed capsule and the NPR material containing the drug is placed into the cavity defined by the PPR material. In some implementations, heat and pressure are applied again to cure and/or bond together the NPR material and the PPR material to form a cohesive structure. In some implementations, a thickness of the PPR material is between 1% and 25% of a total thickness of the drug delivery device.

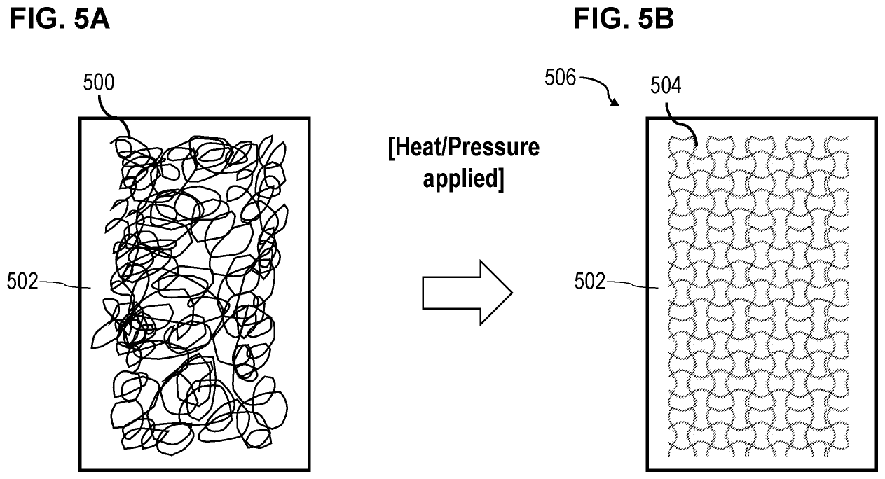

FIGS. 5A-5B show an example of another method of fabricating a drug delivery device with an internal NPR portion. As shown in FIG. 5A, a precursor material 500 is enclosed in a PPR material 502. In some implementations, the precursor material 500 and the PPR material 502 are adhered and/or bonded together. Heat and/or pressure are applied to convert the precursor material 500 into an NPR portion 504 enclosed in the PPR material 502, the NPR portion 504 and the PPR material 502 together forming a drug delivery device 506. The heat and/or pressure are applied in the presence of the drug to be stored into the resulting drug delivery device, e.g., immersed in a solution containing the drug, such that the drug becomes trapped in closed cells of the NPR portion 504. In some implementations, the heat and/or pressure that convert the precursor material 600 into the NPR portion 504 concurrently cure and/or bond together the NPR portion 504 and the PPR material 502, reducing fabrication complexity and cost. In some implementations, the PPR material 502 is itself modified by the heat and/or pressure.

Various modifications will be apparent from the foregoing detailed description. For example, although several implementations in which a drug is stored have been provided, other drugs, liquids, or solids can be stored instead or additionally. For example, various implementations according to this disclosure can be used to store or any other suitable materials that is chemically compatible with material(s) that form the porous NPR structure of drug delivery bodies, and these materials can be stored in liquid, gaseous, or solid states. The stored materials can be a mix of multiple different individual materials. Further, features described above in connection with different implementations may, in some cases, be combined in the same implementation. In some instances, the order of the process steps may differ from that described in the particular examples above.

Accordingly, other implementations are also within the scope of the claims.

What is claimed is:

1. A drug delivery device comprising:
a foam body having a negative Poisson's ratio, the foam body formed of a material configured to dissolve in a biological fluid; and
a liquid drug contained in cells of the foam body, the drug exerting a pressure on walls of the cells that is above atmospheric pressure.

2. The drug delivery device of claim 1, wherein the drug comprises an antihistamine.

3. The drug delivery device of claim 1, wherein the foam body comprises a re-entrant cell structure.

4. The drug delivery device of claim 1, wherein the foam body comprises a disordered, polydisperse foam.

5. The drug delivery device of claim 1, wherein the foam body is a microsphere or a nanosphere.

6. The drug delivery device of claim 1, wherein the foam body has a dimension of less than 100 microns.

7. The drug delivery device of claim 1, wherein the foam body has a dimension of less than 500 nanometers.

8. The drug delivery device of claim 1, wherein at least some of the cells of the foam body are closed cells.

9. The drug delivery device of claim 1, wherein the liquid drug comprises a first liquid drug, and comprising a second liquid drug,
wherein the first liquid drug is contained in a first set of cells of the foam body and the second liquid drug is contained in a second set of cells of the foam body, the cells of the first set located closer to an outer surface of the foam body than the cells of the second set.

10. The drug delivery device of claim 1, wherein the walls of the cells comprise first walls having a first thickness and second walls having a second thickness different from the first thickness.

11. The drug delivery device of claim 1, comprising a coating covering the foam body, the coating dissolvable in the biological fluid and having a positive Poisson's ratio.

12. A drug delivery apparatus comprising:
a capsule defining an interior space, the capsule comprising a material having a positive Poisson's ratio and configured to dissolve in a biological fluid; and
a powder contained in the interior space of the capsule, the powder comprising drug delivery devices, each drug delivery device comprising:
a foam body having a negative Poisson's ratio, the foam body formed of a material configured to dissolve in a biological fluid; and
a liquid drug contained in cells of the foam body, the drug exerting a pressure on walls of the cells that is above atmospheric pressure.

13. The drug delivery apparatus of claim 12, in which the drug delivery device comprises:
first drug delivery devices each comprising a first foam body, in which the first foam bodies have cells comprising walls of a first thickness; and
second drug delivery devices each comprising a second foam body, in which the second foam bodies have cells comprising walls of a second thickness different from the first thickness.

14. The drug delivery apparatus of claim 12, in which the drug delivery device comprises:
first drug delivery devices each comprising a first foam body and a first liquid drug contained in cells of the first foam body; and
second drug delivery devices each comprising a second foam body and a second liquid drug contained in cells of the second foam body, the second liquid drug different from the first liquid drug.

15. The drug delivery apparatus of claim 12, in which the powder comprises drug delivery devices of different sizes.

16. A method of making a drug delivery apparatus, the method comprising:
providing a first body comprising a precursor material;
applying a stimulus to the precursor material, the stimulus causing the precursor material to form a closed-cell porous foam structure having a negative Poisson's ratio; and
encapsulating a fluid drug in the closed-cell porous foam structure such that the fluid drug exerts a pressure on walls of cells of the foam structure that is above atmospheric pressure,
in which the porous foam structure is formed of a material configured to dissolve in a biological fluid.

17. The method of claim 16, wherein the stimulus comprises at least one of heat or pressure.

18. The method of claim 16, wherein the drug comprises an antihistamine.

19. The method of claim 16, comprising:
subsequent to applying the stimulus, dividing the closed-cell porous foam structure into a plurality of drug delivery devices, each drug delivery device having a dimension less than 100 microns.

20. The method of claim 16, comprising forming a coating having a positive Poisson's ratio over the closed-cell porous foam structure.
